(19) United States
(12) Patent Application Publication
(10) Pub. No.: US 2002/0016302 A1
(43) Pub. Date: Feb. 7, 2002

(54) Liposomal antitumor drug and its preparation

(76) Inventors: Grzegorz Grynkiewicz, Lomianki (PL); Jerzy Gubernator, Wroclaw (PL); Arkadiusz Kozubek, Wroclaw (PL); Aleksander Pawel Mazurek, Warsaw (PL)

Correspondence Address: MORGAN & FINNEGAN, L.L.P., 345 PARK AVENUE, NEW YORK, NY, 10154, US

(21) Appl. No.: 09/855,326

(22) Filed: May 15, 2001

(30) Foreign Application Priority Data
Jun. 29, 2000 (PL) P.341123

(51) Int. Cl. A61K 9/127; A61K 31/704

(52) U.S. Cl. 514/34; 424/450

(57) ABSTRACT

The subject of this invention is an antitumor drug from the antracycline family, as well as ways of manufacturing particular liposomal preparations thereof.

### FIELD OF THE INVENTION

The subject of this invention is an antitumor drug from the anthracycline family, as well as ways of manufacturing particular liposomal preparations thereof.

### BACKGROUND OF THE INVENTION

Liposomes are self-closed structures, composed of lipid bilayers, which can entrap active substances in an aqueous solution inside a vessel or incorporate such substances directly into lipid surfaces.

In the last decade liposomes found wide application in pharmacy and medicine. They allow encapsulation of active substances into lipid microspheres and have been applied in particular in cases when an efficacious active substance exhibits severe side effects, which limits its administration. In such cases, efficient encapsulation into liposomes can enhance selectivity of a drug and increase its therapeutic index through improved bioavailability, reduced systemic and organ toxicity or longer half-time of circulation.

A majority of pharmaceutical and medicinal applications is based on small, unilamellar lipid vesicles (“SUV”).

General methods for preparation of liposomes, both unilamellar and multilamellar are known and described inter alia by A. D. Bangham. et al., in J. Mol Biol, 1965, 12: 238; D. D. Lasic, “Liposomes: from Physics to Applications”, Elsevier, Amsterdam, 1995; A. S. Janoff, “Liposomes. Rational Design”, Marcel Dekker, New Jersey, 1998, as well as in patent applications: PCT WO 87/00238 and U.S. Pat. No. 4,558,579.

Among many patent descriptions concerning liposomal formulations of antracycline antitumor antibiotics U.S. Pat. No. 4,419,348 dealing with liposomal Doxorubicin can be taken as an example closely related to the present invention. Liposomal doxorubicin retained antitumor activity, while its characteristic cardiotoxicity was diminished. However, the degree of the active substance encapsulation was fairly low (35-55%) and stability of the liposomal structures was less than satisfactory because of the drug leakage out.

Idarubicin, along with doxorubicin (U.S. Pat. No. 3,590,028) and daunorubicin (U.S. Pat. No. 4,012,284) is one of principal antitumor drugs of the anthracycline family. Its activity in treatment of tumor diseases has been described, inter alia, by G. Capranico et al, Chem. Biol. Interact. 1989; 72:113-123. J. Robert et al. Hematol. Oncol. 1992; 10: 111-116 have shown higher efficiency of idarubicin against certain tumors as compared with doxorubicin and daunorubicin. The same author stated in the said publication that idarubicin is less cardiotoxic than the referred to above drugs.

Taking into account the therapeutic efficacy of idarubicin, one can suppose that liposomal preparation of idarubicin, particularly one featuring a high degree of encapsulation, stability and facile formulation, could become an effective antitumor drug and proving such hypothesis was undertaken as a principal aim of this invention.

### SUMMARY OF THE INVENTION

It is an object of the invention to provide a liposome preparation of idarubicin with encapsulation degree higher than 94%. The liposome preparation preferably contains the lipid components dimyristoylphosphatidylcholine, cholesterol sulfate and cholesterol hemisuccinate in molar proportions 6.5:2.5:1. The liposome preparation also preferably contains 1 weight part of idarubicin for 5 to 30 weight parts of lipids, more preferably 1 part of idarubicin for 15 parts of lipids (w/w).

It is also an object of the invention to provide a method of preparing liposomal idarubicin, wherein a mixture of the active substance and the lipid constituents is made up in an organic solvent and evaporated. The residue is dissolved in cyclohexane, lyophilized and reconstituted by shaking with a buffer solution. The lipid constituents may be natural or synthetic lipids, preferably phospholipids, more preferably derivatives of phosphatidylcholine containing fatty acid residues with C₁₀ to C₂₀ alkyl chains. Cholesterol derivatives, such as cholesterol sulfate and cholesterol hemisuccinate, may be additionally applied. Salts of hydrophobic acids, such as sodium myristoate, sodium palmitate or sodium stearate may also be used as auxiliary compounds. Preferably, the lipid constituents are dissolved in chloroform whereas the active substance and the auxiliary compounds are dissolved in methanol.

In the method of the invention, the preparation may stabilized prior to physical processing. The method may also include extrusion of the liposomal preparation through membranes, preferably having pore size 100 nm, before lyophilization. The liposomal preparation may also be repeatedly subjected to freeze—thaw procedure before lyophilization. Freezing may be achieved by immersion in liquid nitrogen and thawing may be conducted by immersion in a water bath at 40° C. Prior to lyophilization a stabilizing sugar, such as saccharose (sucrose) or trehalose, may be added to the preparation. The amounts of added sugar are preferably 2.5 to 5.0 mg per milligram of lipids when sucrose is used, and 2.5 mg/mg when trehalose is used.

It is also an object of the invention to provide a preparation containing liposomal formulation of idarubicin, containing 10 mg of idarubicin per vial, for a single parenteral administration.

### BRIEF DESCRIPTION OF THE DRAWINGS

FIG. 1. Size distribution of the idarubicin containing liposomes, after extrusion. x-axis: Diameter (nm); y-axis: Size.

FIG. 2 Size stability of idarubicin liposomes, stored as suspension at 4° C. for 6 weeks. x-axis: Days; y-axis: Nanometers.

FIG. 3. Change of encapsulation degree in liposomes stored as suspension at 4° C. for 6 weeks. x-axis: Days; y-axis: Idarubicin in liposomes (%).

FIG. 4. Size distribution of idarubicin containing liposomes after reconstitution. x-axis: Diameter (nm); y-axis: Size.

### DETAILED DESCRIPTION OF THE INVENTION

Idarubicin, idamycin; 5,12-naphthacenedione, 9-acetyl-7-[(3-amino-2,3 ,6-trideoxy-α-L-lyxohexopyranosyl)oxy]-7,8,9,10-tetrahydro-6,9,11-trihydroxy hydrochloride, (7S-cis)-(7S,9)-9-acetyl-7,8,9,10-tetrahydro-6,7,9,11-tetrahydroxy-7-O-(2′,3′,6′-trideoxy-3′-amino-α-L-lyxohexopyranosyl)-5,12-naphthacenedione hydrochloride] is a synthetic anthracycline antibiotic, a formal derivative of Daunorubicin, in which the 4-methoxy group was removed.

Liposomal preparation of idarubicin according to the invention contains the drug substance entrapped in lipid vesicles and the formulation is composed of 1 part of idarubicin and 5 to 30 parts (by weight) of lipids, preferably 1 part of idarubicin for 15 parts of lipids. Pharmaceutically compatible lipids, both: natural and synthetic, such as dimyristoylphosphatidycholine (DMPC) or dipalmitoylphosphatidylcholine (DPPC), acylated phosphoglycerols (for example dimyristoylphosphatidylglycerol, DMPG), esters of cholesterol, such as cholesterol sulfate (ChS) and cholesterol hermisuccinate (ChHS). Salts of hydrophobic acids, like sodium myristoate (SM), sodium stearate (SS) or palmitic acid sodium salt (SP) could also be used as lipid components. Preferred composition of lipids applied as idarubicin carriers consist of dimyristoylphosphatidylcholine (DMPC), cholesterol sulfate (ChS) and cholesterol hemisuccinate (ChHS). Advantageous composition of lipids consists of DMPC:ChS:ChHS in 6.5 to 2.5 to 1 molar ratio.

Liposomal formulation of idarubicin is carried out by dissolving lipid constituents (DMPC or DPPC and ChHS, alternatively DMPG and MS or PS) in chloroform and idarubicin hydrochloride together with ChS in methanol in proportion: 1 part of idarubicin for 5 to 30 parts (by weight) of lipids, preferably 1 to 15 parts. Composition of lipids, which is particularly suitable as idarubicin carrier, consists of DMC, ChS and ChHS in 6.5 to 2.5 to 1 molar ratio. Combined solutions are evaporated to dryness and to the residue cyclohexane is added and the mixture is shaken until homogeneous. Next, the solution is frozen by immersion in liquid nitrogen and subjected to lyophilization. Dry lyophilizate is treated with glycine buffer (pH=6.5) and shaken for 5 min., to obtain a primary liposomal formulation in which entire active substance is contained in the lipid phase.

Primary liposomal formulation of idarubicin can be conveniently stabilized by repeated freezing in liquid nitrogen and thawing in a water bath at 40° C., or alternatively by repeated extrusion through a 100 nm membrane. The extrusion step combines processes of: liposome loading with the active substance, liposome calibration and sterilization of the preparation. Stabilization of the primary liposomal preparation of idarubicin can also be achieved by its lyophilization in the presence of a carbohydrate such as saccharose or trehalose.

According to the invention therapeutically useful liposomal formulation of idarubicin is obtained by repeated extrusion of the primary liposomal preparation through 100 nm pore size membranes or alternatively by repeated freezing in liquid nitrogen and thawing at 40° C. procedure. To such preparation suitable stabilizing sugar (saccharose or trehalose) is added, the mixture is administered to sterile lyophilization vials, 10 mg of idarubicin each, which are subjected to liquid nitrogen freezing, followed by lyophilization. Then vials are capped in sterile conditions under vacuum. The lyophilizate obtained in such a way is stable and can be easily reconstituted with saline before use.

Liposomal preparations of idarubicin, obtained according to the invention are endowed with features considered suitable for pharmaceutical applications. In particular, the obtained liposomes are unilamellar, about 130 nm in diameter and stable for at least 6 weeks when stored at 4° C. Preferred composition of lipids applied as idarubicin carriers, secures high degree of encapsulation, amounting to at least 95%, upon simple shaking of lipid constituents with the active substance. Sterile liposomal lyophilizate formulation can be easily reconstituted with saline or water prior to administration.

### EXAMPLES

A way to obtain liposomal preparation of idarubicin according to the invention is illustrated by, but not limited to, the following examples.

### Example I

20 ml volume, chloroform solution of dimyristoylphosphatidylcholine (10 mg/mL) and cholesterol hemisuccinate (lOmg/mL) were placed in a screw-cap test tube, followed by methanolic solution of cholesterol sulfate (5mg/mL) and idarubicin hydrochloride (2.5 mg/mL) and the solvents were evaporated with a stream of dry nitrogen. The dry residue was treated with cyclohexane (alternatively tert-butanol can be applied) and shaken to dissolution. The obtained solution was frozen by immersion in liquid nitrogen and subjected to lyophilization. To the dry lyophilizate 2 mL of 40 mM glycine buffer (pH=6.5) was added and the test tube was shaken energetically for 5 min., after which time all the active substance was entrapped by the lipids, thus affording the primary liposomal formulation of idarubicin.

Examination of the test tube content after shaking with buffer, under light microscope with magnification 600×, revealed liposomal structures filled up with orange fluid. Supernatant obtained by centrifugation (6 min., 16000 rev./min.) was colorless and did not show UV absorption characteristic for anthracyclines. Also filtration through a Sephadex G-50 column did not show any retention of the active substance, which clearly demonstrated quantitative encapsulation. The active substance content in Sephadex column eluate was determined after breaking down liposomal structures with Triton X-100 detergent, by measuring UV absorption at 498 mn wavelength. Additionally, measurement of phosphorus level (photometric method; with perchloric acid, ammonium molybdate and ascorbic acid) allowed to determine encapsulation as 97.6% with accuracy 0.9.

### Example II

Buffered suspension of liposomes containing the active substance, prepared as described in Example I, was subjected to extrusion through a polycarbonate membrane with pore size 100 nm. Extrusion was carried out in a syringe mini-extruder.

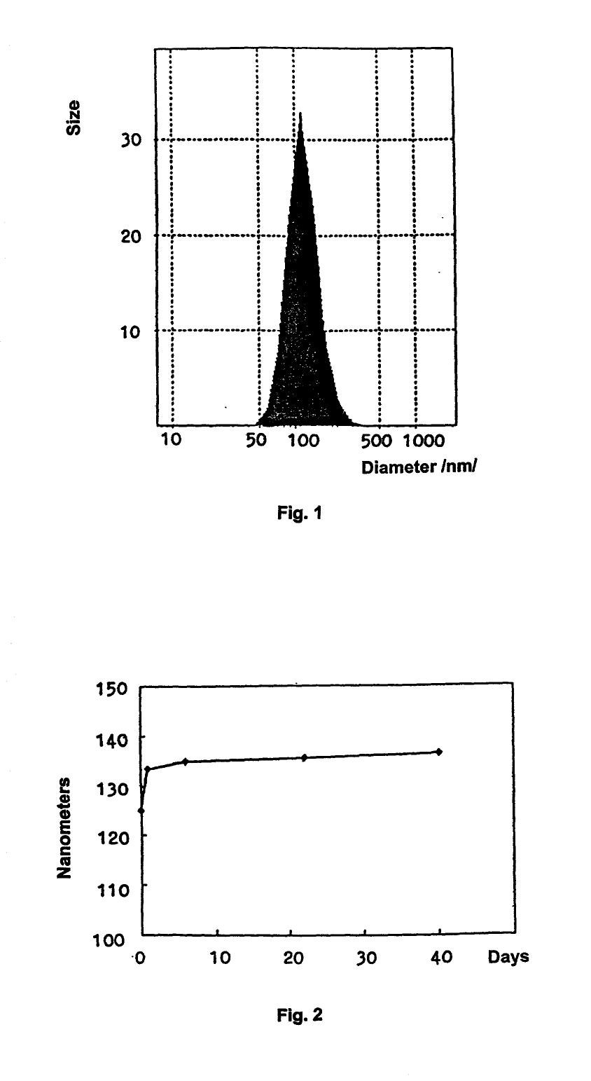

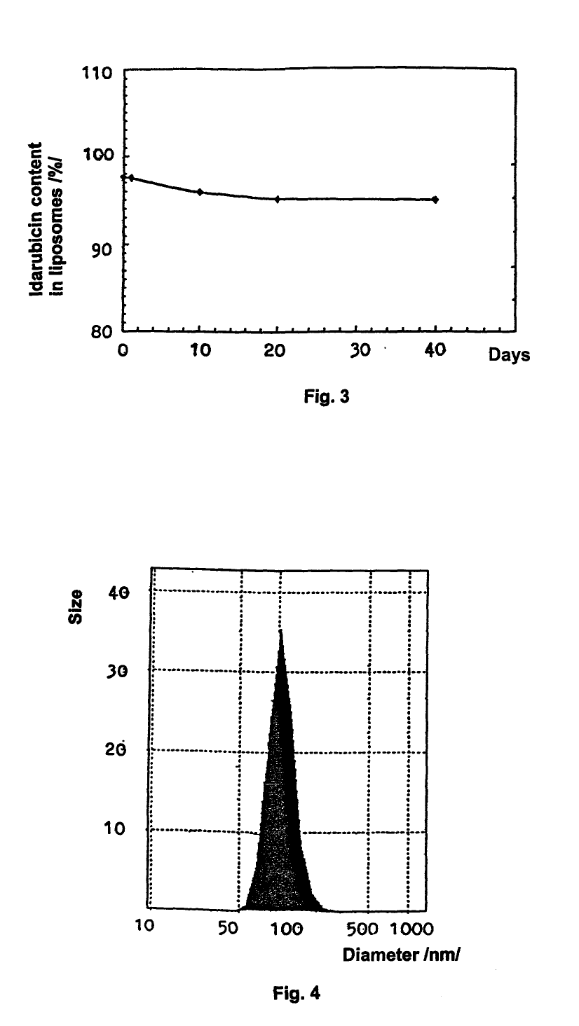

Liposomes thus obtained were unilamellar and uniform in size. After the third extrusion it was found by measurement using photon correlation spectroscopy (“PCS”) that an average liposome size was 120 nm as shown in FIG. 1. Repeated extrusion did not influence or diminish encapsulation of the active substance. Suspension of liposomes was characterized by good stability and could be stored for at least 6 weeks at 4° C. without significant change in size of lipid vesicles (FIG. 2). There was no evidence of the active substance leak-out during the 6 week storage (FIG. 3).

### Example III

To a suspension of liposomes, containing 100 mg of idarubicin hydrochloride in 115 mL of 40 mM glycine buffer (pH=6.5), which was prepared as described in Example I and subsequently subjected to extrusion as described in Example II, sucrose was added, 5 mg for each milligram of the lipid, and the mixture was stirred to dissolution. The lipid thus obtained was distributed to 10 sterile lyophilization vials, frozen through immersion in liquid nitrogen, then placed in lyophilization chamber with plates thermostated at −40° C. Lyophilization was carried out by applying heating gradient 3° C. per hour, until temperature 35° C. was reached, which took approx. 40-45 hours. The vials were the closed under vacuum, in sterile conditions.

It has been demonstrated that the lyophilizate obtained as described above underwent reconstitution in water or physiological solution. In each case, shaking of the lyophilizate with 11.5 ml of saline resulted in reconstitution in approx. 1 min. Liposomes thus obtained had almost unchanged features when compared to the primary preparation obtained by extrusion and their average size, measured by PCS, differed by only 10 nm (FIG. 4). The degree of encapsulation of the active substance measured after reconstitution was 94 (+/−1) %.

### Example IV

Suspension of liposomes, containing 100 mg of idarubicin hydrochloride, in 115 mL of glycine buffer (40 mM, pH=6.5), prepared as in Example I, only DMPC was substituted by equivalent amount of dipalmitoylphosphatidylcholine (DPPC), was submitted to the process of extrusion as described in Example II. Then, sucrose was added (2.5 mg per mg of lipid; sucrose can be replaced by the same amount of trehalose) and the mixture was stirred to homogeneity. Next, the liposomal preparation was distributed to 10 sterile lyophilization vials, which were processed further as described in Example III. Analogous preparation was obtained by taking 5 mg of sucrose for each milligram of the lipid.

It has been demonstrated that both lyophilizates obtained as described above underwent reconstitution in water or physiological solution. In each case, shaking of the lyophilizate with 1.5 ml of saline resulted in reconstitution in approx. 1 min. Liposomes thus obtained had almost unchanged features when compared to the primary preparation obtained by extrusion and their average size, measured by PCS, differed by only 10 nm. The degree of encapsulation of the active substance measured after reconstitution was 94 (+/−1)% and did not depend on the amount of the stabilizing sugar used. Characteristics of the liposomes obtained according to Example IV are presented in Table 1.

TABLE 1
Characteristics of liposomes containing idarubicin after
hydrophylization in presence of stabilizing sugar and after reconstitution in
physiological solution.

| | | Average | | Average | |
|---|---|---|---|---|---|
| | | liposome size | | liposome size | |
| | | after | Polydispersity | after | Polydispersity |
| Experiment | Sugar | extrusion | of preparation | reconstitution | of preparation |
| #4 | Sucrose, | 92 nm | 0.190 | 104 nm | 0.341 |
| | 2.5 mg/mg | | | | |
| #4a | Sucrose, 5 | 106 nm | 0.236 | 103 nm | 0.304 |
| | mg/mg | | | | |
| #4b | Trehalose, | 100 nm | 0 194 | 90 nm | 0.228 |
| | 2.5 mg/mg | | | | |

### Example V

Chloroform solutions of dimyristoylphosphatidylcholine, 10 mg/mL and cholesterol hemisuccinate, 10 mg/mL were placed in a screw-cap test tube, vol. 20 mL, followed by methanolic solutions of cholesterol sulfate, 5 mg/mL and idarubicin hydrochloride, 2.5 mg/mL (1 mL each). The solvents were evaporated in a stream of dry nitrogen, then 2 mL of cyclohexane (alternatively, the same volume of tert-butyl alcohol could be applied) was added and the content was shaken to homogeneity. The obtained mixture was frozen in liquid nitrogen and lyophilized as described above. To the dry lyophilized powder 2 mL of glycine buffer was added (40 mM, pH=6.5) and the content was shaken vigorously for 5 min. Then, the test tube content was subjected to freeze-thaw process (alternative immersion in liquid nitrogen and 40° C. water bath, repeated six times). Examination of the test tube content under light microscope (magnification 600 ×) revealed the presence of liposomal structures, filled with orange content. Supernatant obtained after centrifugation of the content (16 000 rev/min, for 6 min) was colorless. Further examination of the solution, carried out as described in Example I, allowed estimation of encapsulation degree as 97.6+/−0.9%. The size of lipid vesicles was ca. 2 micrometers.

### Example VI

Chloroform solutions of dimyristoylphosphatidylcholine, dimyristoylphosphatidylglycerol and sodium myristoate in weight proportion 6:13:10 (alternatively, sodium salts of palmitic or stearic acids may be used, in the same proportion, instead of myristoate) were placed in a screw-cap test tube, vol. 20 mL, followed by methanolic solution of idarubicin hydrochloride, taken in proportion: lipid to the drug substance=15: 1 (w/w). The solvents were evaporated in a stream of dry nitrogen gas and to the residue 2 mL of cyclohexane was added (or the same volume of tert-butanol) and the content was shaken to dissolution. The mixture thus prepared was frozen in liquid nitrogen and subjected to lyophilization. Solid lyophilizate was treated with 2 mL of glycine buffer and shaken vigorously for five min. After that, the entire active substance was contained in a lipid phase, which was subsequently extruded as described in Example II. Liposomes obtained in the above described way were unilamellar and uniform in size. An average size, as determined by PCS, was 128 nm. Encapsulation degree of the active substance was in range of 95-98%.

### Example VII

Sucrose was added to the liposome suspension, obtained as described in Example VI, prior to lyophilization in amount 1.5 mg per 1 mg of lipids (alternatively trehalose could be used in the same amount). After dissolution, lyophilization was carried out as described in Example III. 2 mL 40 mM glycine buffer (pH 6.5) was added to the dry powder and the mixture was shaken vigorously for 5 min. As a result the liposome suspension was obtained in which all the active substance was contained in lipid phase. Liposomes thus obtained were unilamellar and uniform in size, with average size 182 nm, as determined with the aid of PCS. The attained encapsulation degree was in range of 95-98%.

All of the references and patents referred to herein above are incorporated herein by reference.

The embodiments of the invention, in which an exclusive property or privilege is claimed, are defined as follows:

We claim:

1. A liposome preparation of idarubicin with encapsulation degree higher than 94%.

2. A preparation according to claim 1, containing as lipid components: dimyristoylphosphatidylcholine, cholesterol sulfate and cholesterol hemisuccinate in molar proportions 6.5:2.5:1

3. A preparation according to claim 2, containing 1 weight part of idarubicin for 5 to 30 weight parts of lipids.

4. A preparation according to claim 3, containing 1 part of idarubicin for 15 parts of lipids (w/w).

5. A method of preparing liposomal idarubicin, wherein a mixture of the active substance and the lipid constituents is made up in an organic solvent, evaporated, residue is dissolved in cyclohexane, lyophilized and reconstituted by shaking with a buffer solution.

6. A method according to claim 5, wherein natural or synthetic lipids are applied, in particular phospholipids, derivatives phosphatidylcholine containing fatty acid residues with C₁₀ to C₂₀ alkyl chains.

7. A method according to claim 6, wherein cholesterol derivatives, such as cholesterol sulfate and cholesterol hemisuccinate, are additionally applied.

8. A method according to claim 6, wherein salts of hydrophobic acids, such as sodium myristoate, sodium palmitate or sodium stearate are used as auxiliary compounds.

9. A method according to claim 5, wherein the lipid constituents are dissolved in chloroform whereas the active substance and auxiliary substances are dissolved in methanol.

10. A method according to claim 5, wherein the preparation is stabilized prior to physical processing.

11. A method according to claim 10, wherein extrusion through membranes, pore size 100 nm, is carried out before lyophilization.

12. A method according to claim 10, wherein before lyophilization the preparation is repeatedly subjected to freeze - thaw procedure.

13. A method according to claim 12, wherein freezing is achieved by immersion in liquid nitrogen and thawing is conducted by immersion in a water bath at 40° C.

14. A method according to claim 10, wherein prior to lyophilization a stabilizing sugar, such as sucrose or trehalose, is added to the preparation.

15. A method according to claim 14, wherein amounts of added sugar are 2.5 to 5.0 mg per milligram of lipids in case of sucrose and 2.5 mg/mg for trehalose.

16. A preparation containing liposomal formulation of idarubicin, containing 10 mg of idarubicin per vial, for a single parenteral administration.
